(19) United States
(12) Patent Application Publication
(10) Pub. No.: US 2010/0298838 A1
(43) Pub. Date: Nov. 25, 2010

(54) SURGICAL SCREWDRIVER

(75) Inventor: Carmen Walters, Carlsbad, CA (US)

Correspondence Address: ALPHATEC SPINE, INC., 5818 EL CAMINO REAL, CARLSBAD, CA, 92008, US

(73) Assignee: Alphatec Spine, Inc., Carlsbad, CA (US)

(21) Appl. No.: 12/783,321

(22) Filed: May 19, 2010

Related U.S. Application Data
(60) Provisional application No. 61/179,500, filed on May 19, 2009.

(51) Int. Cl. A61B 17/58; A61B 17/88

(52) U.S. Cl. 606/104; 81/58.1

(57) ABSTRACT

A surgical screwdriver having two drive modes of operation, an axial mode and a ratcheting mode. The screwdriver includes a handle having a base, a shaft rotatably coupled to the handle proximate the base, the shaft having one or more grooves along a length, an outer sleeve having a ratchet mechanism and an internal bore configured to receive the shaft, and a distal shaft having a proximal end coupled with the outer sleeve and a distal working end configured to couple with a screw. When the screwdriver is in the axial mode of operation, the shaft is moved in axial translation with the outer sleeve and the ratchet mechanism engages the grooves to rotate the working end. When the screwdriver is in the ratcheting mode of operation, the handle base is coupled to outer sleeve and rotation of the handle activates the ratchet mechanism to rotate the working end.

### CROSS-REFERENCE TO RELATED APPLICATIONS

The present application claims priority to U.S. Provisional Patent Application Nos. 61/179,500, filed May 19, 2009, the disclosures of which is incorporated herein by reference in its entirety.

### BACKGROUND OF THE INVENTION

### Field of the Invention

Embodiments of the present application are directed to a surgical screwdriver and in particular, to a surgical screwdriver having a mechanism that allows for rapid advancement of a bone screws using axially directed force.

Surgical intervention is often required to treat injuries and deformities of the spine. Often, to treat injuries and deformities, a large number of screws may need to be inserted into the spine of a patient. Due to the large number of screws, conventional surgical screw drivers and methods often result in very long operations and surgeon fatigue.

It is desirable to have an improved surgical screw driver and associated methods that reduce operation time and surgeon fatigue.

### SUMMARY

Embodiments of the present application are directed to an improved surgical screwdriver having two drive modes of operation, and in particular, to a screwdriver incorporating a mechanism that facilitates delivering a screw in a bone during surgery. According to some embodiments, the screwdriver can be used in two separate modes, an “axial” (pumping) mode and a “ratcheting” (standard) mode. A screwdriver capable of axial mode may be known as a push/pull type screwdriver or Yankee screwdriver. These modes can be activated quickly and can be interchanged during the installation of a screw throughout a surgical procedure. The two modes provide surgeons with increased flexibility and control over surgical procedures, and can reduce the operative time and fatigue experienced by the surgeon. In some embodiments, the initial mode (first mode) is an axial mode to quickly rotate the screw, followed by the ratcheting mode for the final rotations of the screw. Is some embodiment, the modes may be done oppositely, with the first mode being the ratcheting mode and the second mode being the axial mode.

### BRIEF DESCRIPTION OF THE DRAWINGS

FIG. 1 illustrates a perspective view of a surgical screwdriver in a first position according to some embodiments of the present application.

FIG. 2 illustrates a perspective view of the surgical screwdriver in FIG. 1 in a second position.

### DETAILED DESCRIPTION OF THE EMBODIMENTS

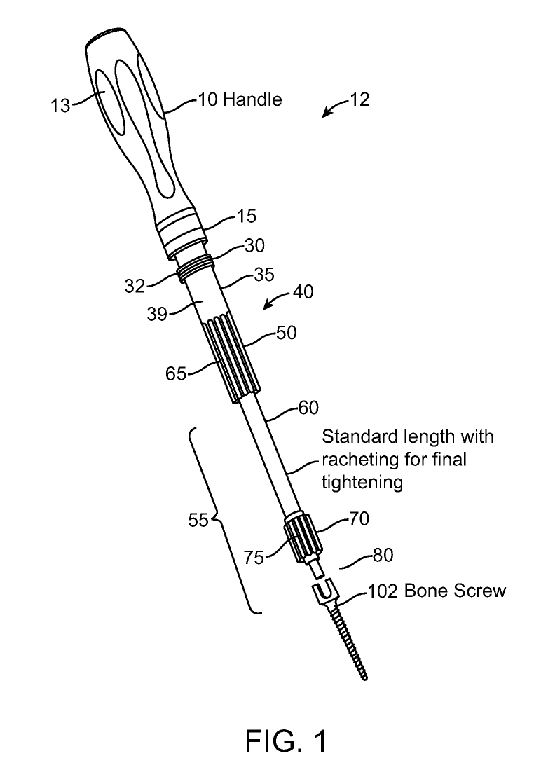

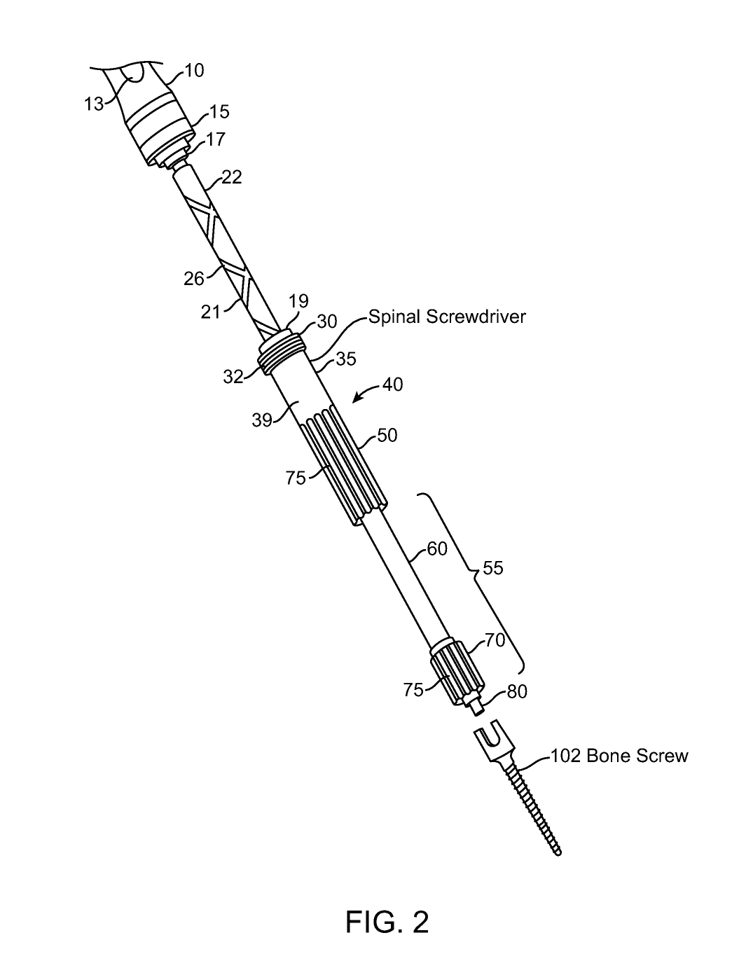

FIG. 1 illustrates a perspective view of a screwdriver 12 in a first position showing a ratcheting mode according to some embodiments of the present application, while FIG. 2 illustrates a perspective view of the screwdriver 12 of FIG. 1 in a second position showing an axial driving mode. The screwdriver 12 comprises a handle 10, an extendable shaft 22 (shown in FIG. 2), a locking collar 30, an outer sleeve 40, and a distal portion 55. The distal portion 55 includes a distal shaft 60, a handle portion 70 and a working end 80. The distal shaft 60 can comprise a smooth cylindrical shaft in some embodiments.

In some embodiments, the screwdriver 12 can be employed in a ratcheting mode. The ratcheting mode can be used with the extended shaft 22 in a retracted position as shown in FIG. 1. For example, the screwdriver handle 10 can be positioned in its most downward or distal position and can be locked by the locking collar 30. In this mode, the screwdriver 12 can serve as a ratcheting screwdriver, whereby, in some embodiments, rotation of the handle 10 can result in either clockwise or counterclockwise rotation of the working end 80 to drive a bone screw 102 into a surgical location. The ratcheting arrangement permits the handle 10 to rotate with the working end 80 in one direction and to rotate relative to the working end 80 in the opposite direction. In some embodiments, in the ratcheting mode, the screwdriver 12 can also be kept in a neutral position, whereby the working end 80 is fixed, e.g., without any rotation relative to the working end 80 such that the screwdriver may serve as a conventional screw driver.

In the axial mode position shown in FIG. 2, the screwdriver handle 10 can be extended upwardly or proximally (e.g., away from its most downward position closest to the outer sleeve 40). Such movement can expose at least a portion of the extendable shaft 22. In the axial mode, the handle 10 can be pushed downwardly (e.g., towards its most downward position closest to the outer sleeve 40) along a longitudinal axis of the screwdriver 12. The axial translation of the handle 10 caused by the downward motion can result in either a clockwise or counterclockwise rotation of the working end 80. The direction of rotation of the working end 80 is controlled by a lug 39 of selector mechanism 35. Such rotation can be used to drive a bone screw 102 into a surgical location, e.g., a vertebra. Also, in the axial mode, the rotation of the working end 80 results without any corresponding rotation of the handle 10, such that the surgeon need not twist his or her hand during axial driving.

In some embodiments, in the axial mode, after being pushed downwardly, the handle 10 can repeatedly be placed in an upward position (e.g., by pulling upwardly on the handle or with the assistance of a biasing mechanism), and once again pushed downwardly to cause additional rotation of the working end 80 of the screwdriver 12. This can be repeated one or more times until a bone screw 102 is driven into a surgical location. A surgeon performing a surgery therefore has at least two options for driving a bone screw into a bone, e.g., a ratcheting or standard mode option involving rotation of the handle and an axial mode option involving axial translation or pumping of the handle. Providing two driving modes enables the surgeon to use the axial mode for a majority of the advancement of the screw, e.g., nearly the entire advancement, while permitting the surgeon to switch to direct or ratcheting mode for a short portion of the screw advancement. In some techniques, this enables the surgeon to quickly advance most of the length of a shank of the screw 102 thus reducing procedure time. These structures provide greater control over the surgical procedure.

The handle 10 of the screwdriver 12 has a plurality of grooves 13 and a cylindrical base 15. In some embodiments, the plurality of grooves 13 are disposed evenly around the circumference of the handle 10 surface, and serve as gripping surfaces. The cylindrical base 15 of the handle 10 includes a first mating surface 17 that is capable of engagement with a second mating surface 19 of the locking collar 30 when the handle 10 is placed in a downward position (e.g., a downward-most position).

In some embodiments, the extendable shaft 22 is coupled with the cylindrical base 15 of the handle 10 (see FIG. 2). In other embodiments, the extended shaft 22 can be affixed to a different portion of the screwdriver 12 (e.g., between the outer sleeve 40 and working end 80). In some embodiments, the extended shaft 22 includes one or more helical structures, e.g., helical grooves 26, 27, that control the rotational direction (e.g., either clockwise or counterclockwise) of the working end 80 of the screwdriver when in use in the axial mode. As shown in FIG. 2, the extended shaft 22 includes two opposing helical grooves 26, 27, one for rotation in a clockwise direction, and another for rotation in a counterclockwise direction, when used in association with an axial mode (discussed below). As the extendable shaft 22 is coupled with the handle 10, pushing and pulling the handle 10 will result in a corresponding downward or upward movement along a longitudinal axis of the extended shaft 22. Repeated pushing and pulling of the handle 10 will result in a rotating action that drives and rotates a bone screw, which can be enhanced by a spring member provided at the base of the extendable shaft 22 in some embodiments.

In some embodiments, the locking collar 30 has a plurality of grooves 32 that extend axially. The locking collar 30 is configured to receive the cylindrical base 15 of the handle 10 when the handle 10 is positioned distally. When the handle 10 is in its most downward position (as shown in FIG. 1), the locking collar 30 can assume either a “closed” or locked position preventing movement of extendable shaft in either direction by engaging a ‘key’ feature or the like or an “open” or unlocked position with respect to the handle 10. The locking collar 30 can be placed in a locked position or an unlocked position by rotating the locking collar 30 clockwise or counter clockwise.

In some embodiments, when the handle 10 is in its most downward position and the locking collar 30 is placed in a locked position, the screwdriver can serve as a ratchet screwdriver. In some embodiments, when the handle 10 is in its most downward position and the locking collar 30 is placed in an unlocked position, the handle 10 of the screwdriver remains subject to upward and downward axial translation. Such axial translation preferably is converted into rotation motion of the working end 80, thereby providing for axially driving of a bone screw. This feature provides an advantage over conventional bone screwdrivers, as the force that can be applied to drive a screw into a bone can be from an axial force, rather than a conventional rotational force or torque. Rotational force or torque can be time consuming and can cause surgeon fatigue.

In some embodiments, the outer sleeve 40 housing a ratchet mechanism and an internal bore. In some embodiments, the ratchet mechanism comprises one or more ratchet wheels, preferably two ratchet wheels with associated pawls, for assisting in clockwise and counterclockwise rotation of the working end 80 of the screwdriver 12. The ratchet mechanism works in conjunction with a lug 39 of a selector mechanism 35 to control the direction of rotation of the working end 80.

In one embodiment, the selector mechanism 35 can be placed in three different settings, e.g., forward, reverse and neutral, by adjusting the lug 39. When the lug 39 is placed in a forward setting, clockwise rotation of the handle 10 produces a corresponding clockwise directed torque at the working end 80 of the screwdriver 12 (e.g., to drive in a bone screw with a right-handed thread). When the lug 39 is placed in a reverse setting, counterclockwise rotation of the handle produces a corresponding counterclockwise directed torque at the working end 80 of the screwdriver 12 (e.g., to remove a bone screw with a right-handed thread). When the lug 39 is placed in a neutral position, the working end 80 of the spiral screwdriver 12 will not rotate, and the screwdriver can serve as a manual screwdriver.

In some embodiments, the outer sleeve 40 further comprises a first gripping surface 50. The first gripping surface 50 comprises a plurality of vertical flutes or protrusions 65 that provide a stable, grippable (e.g., frictional) surface when held. This is beneficial, particularly when the spiral screwdriver is being used in an axial mode, as the surgeon can hold the outer sleeve 40 with one hand while holding the handle 10 to provide a push pumping action.

The distal shaft 60 preferably is disposed distally of and, in some cases, immediately adjacent to the outer sleeve 40. While in some embodiments, the distal shaft 60 has an outer diameter that is greater than the outer diameter of the outer sleeve 40, in other embodiments, the distal shaft 60 has an outer diameter that is less than the outer diameter of the outer sleeve 40. In some embodiments, the distal shaft 60 serves as a second gripping surface that can be held in one hand while the surgeon holds another portion (e.g., the handle 10) of the screwdriver 12. In some embodiments, the gripping surfaces will be ergonomically designed to provide maximum comfort to the surgeon during operation.

The length of the distal shaft 60 preferably corresponds to the nature of the procedure to be performed, e.g., for performing surgical procedures in patients of different sizes. In some embodiments, the length of the distal shaft 60 is fixed, while in other embodiments, the length can be adjustable. In some embodiments the distal shaft may be a chuck or similar connection such that a variety of sizes and configurations of working tips may be used.

In some embodiments, the handle portion 70 is formed contiguously with the distal shaft 60. The handle portion 70 comprises a plurality of vertical flutes or protrusions 75 that serve as a gripping surface. In some embodiments, the handle portion 70 has an outer diameter that is greater than the outer diameter of the distal shaft 60.

The working end 80 preferably extends beyond a distal portion of the handle portion 70. The working end 80 is capable of rotation in both the ratcheting mode or in the axial mode. The direction of rotation of the working end 80 can be controlled by the lug 39 of the selector mechanism 35. The working end 80 is configured to be adapted to the spinal screwdriver 12 to drive in and rotate a bone screw 102 into a desired surgical location (e.g., a pedicle, ilium or sacrum, among other possible locations).

As portions of the screwdriver, including the working end 80, the handle portion 70 and the distal shaft 60, may have to be maintained in a patient for an extended period of time and/or may have to be re-inserted multiple times into a patient to drive multiple bone screws into a patient at various surgical locations, those portions preferably are comprised of biocompatible materials that are sterile or capable of sterilization. Examples of suitable materials for the distal shaft 60 and the handle portion 70 include substantially rigid plastic materials including alpha-olefins including polyethylene and polypropylene, copolymers of styrene-acrylonitrile or acrylonitrile-butadiene-styrene, and more preferably, various metals, such as surgical-grade stainless steel, titanium, alloys and composites. In addition, a protective layer, such as a silicone sheath, can be provided over any material or portion to be protected. Examples of suitable materials for the working end 80 comprise metals such as surgical-grade stainless steel, titanium, alloys and composites.

In operation, the spinal screwdriver 12 can be used in a ratcheting mode, an axial mode or a combination of both modes during the same procedure. Allowing for both modes of operation provides for a reduction in surgeon fatigue during operation and provides for an overall reduced surgical time.

In axial mode spinal screwdriver advances bone screw with minimal force and axial strokes. To deliver the remainder of the bone screw shaft 102 into a desired surgical location, the spinal screwdriver 12 can be shifted to ratcheting mode. In some embodiments, advancement cannot continue beyond the thread length of the bone screw or base of the bone screw body.

It will be apparent to those skilled in the art that various modifications and variations can be made in the present invention without departing from the scope or spirit of the invention. Thus, it is intended that the present invention cover the modifications and variations of this invention provided that they come within the scope of the appended claims or their equivalents.

What is claimed is:

1. A surgical screwdriver having two drive modes of operation comprising:
a handle having a base;
a shaft rotatably coupled to the handle proximate the base, the shaft having one or more grooves along a length;
an outer sleeve having a ratchet mechanism and an internal bore configured to receive the shaft;
a distal shaft having a proximal end coupled with the outer sleeve and a distal working end configured to couple with a screw;
wherein in an axial mode of operation the shaft is moved in axial translation with the outer sleeve and the ratchet mechanism engages the grooves to rotate the working end, and in a ratcheting mode of operation the handle base is coupled to outer sleeve and rotation of the handle activates the ratchet mechanism to rotate the working end.

2. The screwdriver of claim 1, wherein the ratchet mechanism includes a selector mechanism configured to change the mode of operation between the axial mode and the ratcheting mode.

3. The screwdriver of claim 2, wherein in the ratcheting mode of operation the selector mechanism includes a ratcheting screwdriver position whereby rotation of the handle results in either a clockwise or counterclockwise direction.

4. The screwdriver of claim 2, wherein in the axial mode of operation the selector mechanism only allows clockwise or counterclockwise rotation of the working end during a downward motion of the handle toward the outer sleeve.

5. The screwdriver of claim 4, wherein the selector mechanism further includes a lug configured to engage the one or more grooves, the lug being movable from a first engagement position configured to rotate the working end in a clockwise rotation during axial translation of the shaft, and a second engagement position configured to rotate the working end in a counterclockwise rotation during axial translation of the shaft.

6. The screwdriver of claim 1, wherein the one or more grooves are helical grooves on an external surface of the shaft.

7. The screwdriver of claim 1, further comprising a locking collar configured to lock the handle to the outer sleeve.

8. The screwdriver of claim 1, wherein the one or more grooves are opposing helical grooves on an external surface.

9. The screwdriver of claim 1, wherein the shaft includes a biasing mechanism.

10. A surgical screwdriver comprising:
a handle having a plurality of grooves distributed about its surface and a cylindrical base;
an extendable shaft affixed to the cylindrical base of the handle, the extendable shaft comprising opposing helical grooves on its external surface and an internal biasing mechanism;
a locking collar comprising a plurality of circumferential grooves and configured to receive the cylindrical base of the handle;
an outer sleeve housing a ratchet mechanism and an internal bore, the outer sleeve comprising a selector mechanism and a first gripping surface, the selector mechanism comprising a lug configured to control the ratchet mechanism, the first gripping surface comprising a plurality of vertical protrusions, wherein the extended shaft is configured to be slideably inserted through the bore of the outer shaft;
a distal shaft formed contiguously with the outer shaft, the distal shaft having an outer diameter less than the outer diameter of the outer sleeve, wherein the distal shaft provides a second gripping surface;
a lower handle portion formed contiguously with the distal shaft, the lower handle portion having an outer diameter greater than the outer diameter of the distal shaft, the lower handle portion comprising a plurality of vertical protrusions to provide a third gripping surface; and
a working end extending beyond the base of the lower handle portion, the working end configured to be attachable to a screw member.

11. A method of inserting a bone screw in a bone during surgery, comprising:
providing a bone screw;
attaching the bone screw to the working end of a screwdriver having two drive modes of operation, the screwdriver comprising:
a handle engagably coupled to the working end;
wherein, in an axial mode of operation the handle is configured for upward and downward movement along a longitudinal axis to rotate the working end; and
in a ratcheting mode of operation the handle is configured to rotate to rotate the working end;
selecting the axial mode of operation by placing the handle in an upward position away and pushing the handle one or more times from the upward position to a downward position to rotate the working end and start to advance the bone screw into the bone; and
selecting the ratcheting mode of operation and rotating the handle to rotate the working end until the bone screw is fixed in the bone.

12. The method of claim 11, wherein the screwdriver includes a selector mechanism configured to change the mode of operation between the axial mode and the ratcheting mode.

13. The method of claim 12, wherein the handle is attached to an extendable shaft engagably coupled to an outer sleeve housing, the outer sleeve housing having an internal bore for receiving the extendable shaft wherein when the handle is pushed downwardly along the longitudinal axis from an upward position rotates the working end.

14. The method of claim 13, wherein the selector mechanism further includes a lug configured to engage the one or more grooves, the lug being movable from a first engagement position configured to rotate the working end in a clockwise rotation during axial translation of the shaft, and a second engagement position configured to rotate the working end in a counterclockwise rotation during axial translation of the shaft.

15. The method of claim 11, wherein the screwdriver includes a ratchet mechanism configured to couple with the handle and control the direction of rotation the working end.
